(19) United States
(12) Patent Application Publication
(10) Pub. No.: US 2008/0038832 A1
(43) Pub. Date: Feb. 14, 2008

(54) Method for Producing Molecularly Imprinted Polymers for the Recognition of Target Molecules

(75) Inventors: Borje Sellergren, Dortmund (DE); Andrew J. Hall, Sunderland (GB)

Correspondence Address: SCHWEGMAN, LUNDBERG & WOESSNER, P.A., P.O. BOX 2938, MINNEAPOLIS, MN, 55402, US

(73) Assignee: MIP Technologies AB

(21) Appl. No.: 11/665,239

(22) PCT Filed: Oct. 12, 2005
(86) PCT No.: PCT/SE05/01515

§ 371 Date: Oct. 17, 2007

(30) Foreign Application Priority Data
Oct. 12, 2004 (DE) 10 2004 049 805.9

(51) Int. Cl. G01N 33/00; C08G 73/00

(52) U.S. Cl. 436/106; 528/367

(57) ABSTRACT

The present invention relates to a method of preparing a molecularly imprinted polymer (MIP), which are used for the recognition of target molecules comprising: co-polymerising at least one functional monomer and at least one cross-linking monomer in the presence of at least one template, wherein oxyanions are used as template and the steric and/or electronic structure of the template is at least partly analogous to the target molecule. The target molecules may be nitro-containing compounds, such as nitroaromatic compounds, or lactones. MIPs selective for explosive nitro-aromatic substances may be produced without handling these hazardous compounds. The invention further relates to a method of determining if a sample contains nitro-containing compounds, such as nitroaromatic compounds, or lactones, MIPs selective for nitro-containing compounds and/or lactones, especially nitro-aromatic compounds and a kit, comprising a molecularly imprinted polymer selective for nitro-aromatic compounds and/or lactones. The invention also relates to use of isosteric and/or isoelectronic oxyanions for the production of MIPs for recognition of nitro-containing compounds, especially nitroaromatic compounds, and lactones.

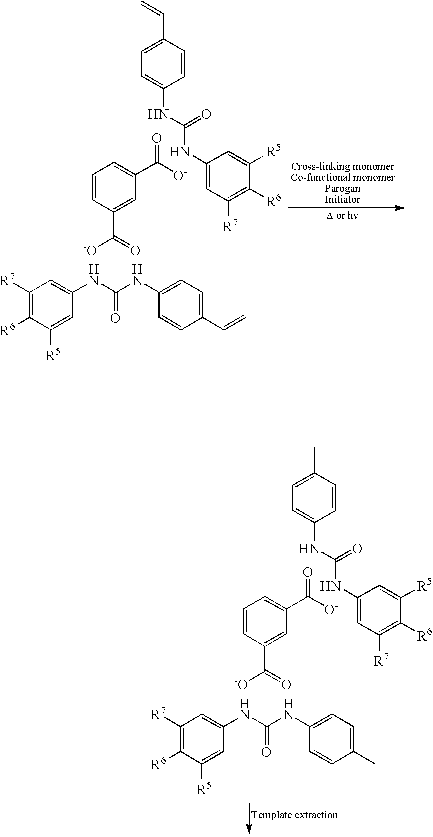

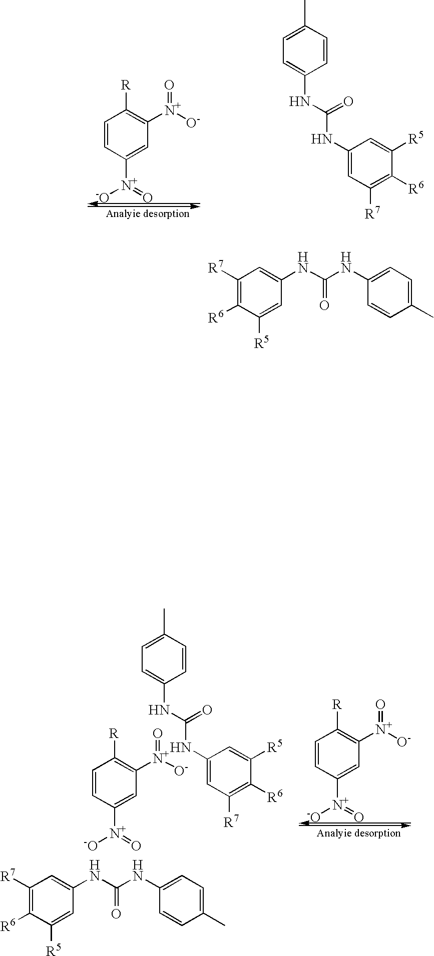

The present invention relates to a method of preparing molecularly imprinted polymers (MIPs), which are used for the recognition of target molecules, comprising co-polymerising at least one functional monomer and at least one cross-linking monomer in the presence of at least one template, wherein oxyanions are used as template and the steric and/or electronic structure of the template is at least partly analogous to the target molecule. The target molecules may be nitro-containing compounds, such as nitro-aromatic compounds, or lactones. MIPs selective for explosive nitro-aromatic substances may be produced without having to handle these hazardous compounds.

The invention further relates to a method of determining whether a sample contains nitro-containing compounds, such as nitro-aromatic compounds, or lactones. Further, the invention regards MIPs selective for nitro-containing compounds and/or lactones, especially nitro-aromatic compounds and a kit, comprising a MIP selective for nitro-aromatic compounds and/or lactones.

The invention also relates to use of isosteric and/or isoelectronic oxyanions for the production of MIPs for the recognition of nitro-containing compounds, especially nitro-aromatic compounds, and lactones.

### PRIOR ART

Today, there are increasing demands concerning product quality and purity. Therefore, it is desirable that more selective, sensitive and fast methods of analysis become available. Especially regarding areas such as environmental control, food control, doping tests, medicine, chemistry, pharmacy, food technology and biotechnology, and particularly in diagnostics and drug development, there is an urgent need for efficient separation and purification methods. Alternatively, it is also possible to use specific sensors for the detection of molecules. In this way it is possible to perform a specific and selective identification of definite molecules or groups of molecules.

Especially in the measurement of harmful substances, for instance in environmental analysis, it is very important to find efficient methods for detection and/or identification. Harmful substances are, for instance, nitrogenous substances, such as nitro-containing compounds, which may also include explosive substances. In the identification of explosive materials, the sensitivity and selectivity of the method are very important. The identification of such substances has to be performed well below the ppm level. This is complicated by the fact that these substances generally have a very low vapour pressures. Also in order to identify and to classify harmless compounds with similarly low vapour pressures, for example odour substances, the method has furthermore to be very selective.

In order to trace explosive substances specially trained dogs are commonly used. The ability of the dogs to identify these substances lies in the ppt range. However, a disadvantage with the use of animals is the high costs for the training of the dogs, which can take many years. There are also a number of logistical problems leading to further costs, such as the need for a human handler, taking care of the animal and transporting the animal and handler to the desired location. The detection of nitrocompounds in the ppt range poses thus a great challenge for the chemical sensor technique. In order to secure a high specificity and selectivity of the gas sensor systems and at the same time obtain a cost reduction, the use of molecularly imprinted polymers (MIPs) offers an attractive alternative. The use of MIPs has proven to be an effective method for selective enrichment of analytes. Further, gaseous analytes may also be separated and enriched to measurable amounts.

The molecular imprinting method was first used for the selective recognition of small molecules some years ago. “Non-covalent” molecularly imprinted materials are used today for the recognition of different small molecules, such as therapeutics, sugars, nucleotides, pesticides, steroids, peptides and hormones.

The molecular imprinting method usually includes the following steps:

1. The target molecule, or a molecule which has a similar steric structure as the target molecule, is used as a template and allowed to interact with given functional monomers.

This step makes use of non-covalent self-assembly of the template with the functional monomer(s) prior to polymerisation.

2. The resulting template-monomer complexes are then polymerised into a 3D-network polymer, by co-polymerisation with a network monomer.

The polymerisation may be performed in the presence of a pore-forming solvent called a porogen. In order to stabilise the electrostatic interactions between the functional monomers and the template, the porogen is often chosen to be aprotic and of low to moderate polarity

3. Subsequently, the template is released from the polymer matrix.

This results in a polymer with specific cavities, which have size, shape and functional group complementarity to the template. Thus, the polymer displays very strong affinity for the template and related compounds.

MIPs formed in this way can then be used for chromatographic separation of different substances in column chromatography, in solid phase extraction or as receptor films in chemical sensors.

The search for suitable templates represents a particular problem for the preparation of MIPs. It is possible to use the target molecule itself as template, but only as long as the target molecule does not react with the functional and/or cross-linking monomers and is stable under the polymerisation conditions. Furthermore, target molecules can often not be used because of their harmful properties, such as explosiveness or toxicity. Target molecules can also inhibit or prevent the polymerisation. Further, they may be insoluble or only sparingly soluble in the polymerisation mixture. All these disadvantages have to be considered when searching for a template.

The majority of templates used so far exhibit moderate to high solubility in the resulting polymerisation media and they (or their structural analogues) can therefore be used directly in the conventional procedure. However, for the following reasons, a structural analogue to the target molecule is commonly preferred as template.

1. The target is unstable under the polymerisation conditions or inhibits the polymerisation.

2. The target is not available in sufficient quantities to make imprinting worthwhile.

3. To avoid template bleeding, i.e. the bleeding of low levels of template from the MIP, thus precluding SPE applications in trace analysis.

4. The target is insoluble or poorly soluble in the pre-polymerisation mixture.

5. The target is toxic or hazardous in other ways, e.g. explosive.

With regards to nitroaromatic compounds, limitations 1, 3 and 5 apply. This effectively precludes the use of these targets as templates in the generation of the MIPs. It is therefore urgent to find target analogues, which can function as templates for this class of analytes. Furthermore, it is also urgent to find functional monomers that can provide relatively strong interactions with the target analyte in the application matrix. This is unlikely to be the case with the (commercially available) functional monomers most commonly used in non-covalent imprinting protocols.

Explosive materials in particular, are not suitable as templates. Therefore templates and methods for producing MIPs that do not demand the use of harmful substances are preferred.

According to the prior art of this technique a method (WO 01/77664) is known by which a porphyrin derivative and a target molecule (an explosive chemical) are used. The porphyrin derivative is here complexed with TNT and subsequently the ligand-template complex immobilised in a polymer matrix in order to reduce or eliminate the explosiveness of TNT. A disadvantage, however, is that the use of explosive materials (TNT) in the preparation of such MIPs cannot be avoided.

Consequently, the purpose of the invention is to provide a method with which MIPs, which are suitable for the recognition of, for instance, explosive target molecules, can be produced without using nitro-containing compounds as templates.

The use of nitro-containing compounds as templates in free radical polymerisation is unfavourable as it leads to an incomplete polymerisation, which implies a reduction in the quality of the MIPs. The risk of a possible explosion in the handling of nitro-aromatic compounds has also to be avoided.

In order to solve this problem the invention suggests a method by which isoteric and/or isoelectronic template analogues are used and by which the steric structure of the template is at least partly analogous to the structure of the target molecule, i. e. nitro-aromatic compounds and/or lactones,

### SUMMARY OF THE INVENTION

The present invention relates to a method of preparing molecularly imprinted polymers (MIPs), which are used for the recognition of target molecules, comprising co-polymerising at least one functional monomer and at least one cross-linking monomer in the presence of at least one template, wherein oxyanions are used as template and the steric and/or electronic structure of the template is at least partly analogoss to the target molecule. The target molecules may be nitro-containing compounds, such as nitroaromatic compounds, or lactones. MIPs selective for explosive nitro-aromatic substances may be produced without handling these hazardous compounds.

The invention further relates to a method of determining whether a sample contains nitro-containing compounds, such as nitro-aromatic compounds, or lactones. Further, the invention regards MIPs selective for nitro-containing compounds and/or lactones, especially nitro-aromatic compounds and a kit, comprising a MIP selective for nitro-aromatic compounds and/or lactones.

The invention also relates to use of isosteric and/or isoelectronic oxyanions for the production of MIPs for recognition of nitro-containing compounds, especially nitro-aromatic compounds, and lactones.

The following figures describe the invention:

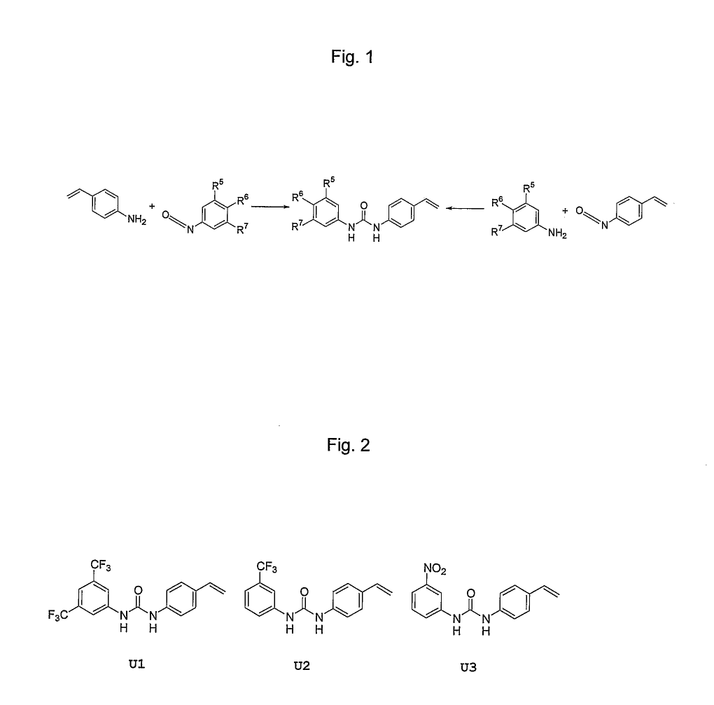

FIG. 1 Production of polymerisable 1,3-disubstituted monoureas;

FIG. 2 Non-limiting examples of strong-binding 1,3-disubstituted mono-urea functional monomers;

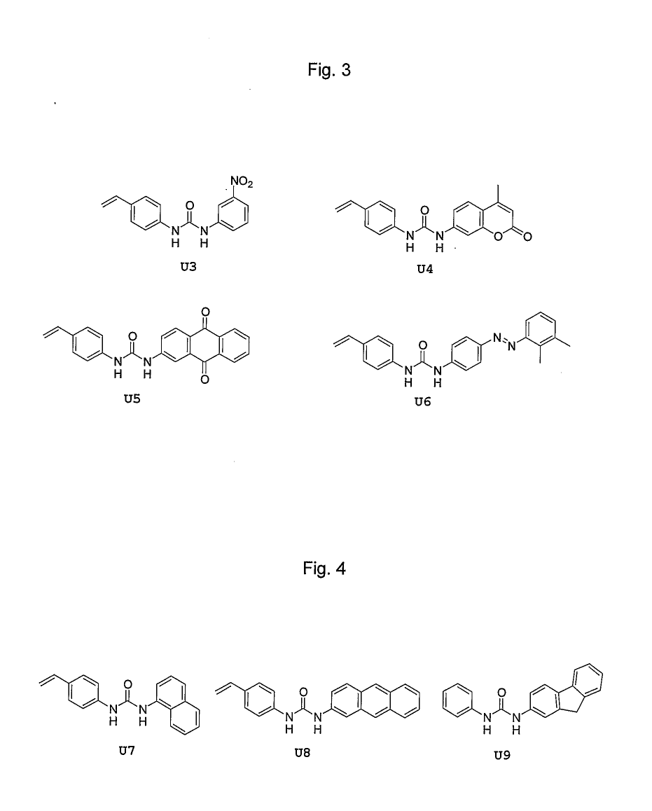

FIG. 3 Non-limiting examples of chromogenic 1,3-disubstituted mono-urea functional monomers;

FIG. 4 Non-limiting examples of flurogenic 1,3-disubstituted mono-urea functional monomers;

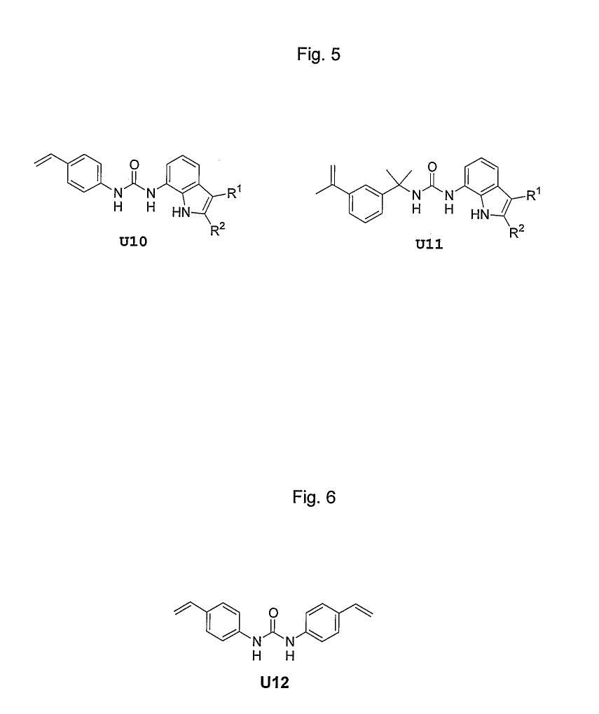

FIG. 5 Non-limiting examples of flurogenic, indole-containing 1,3-disubstituted mono-urea functional monomers;

FIG. 6 Non-limiting examples of a cross-linking 1,3-disubstituted mono-urea functional monomer;

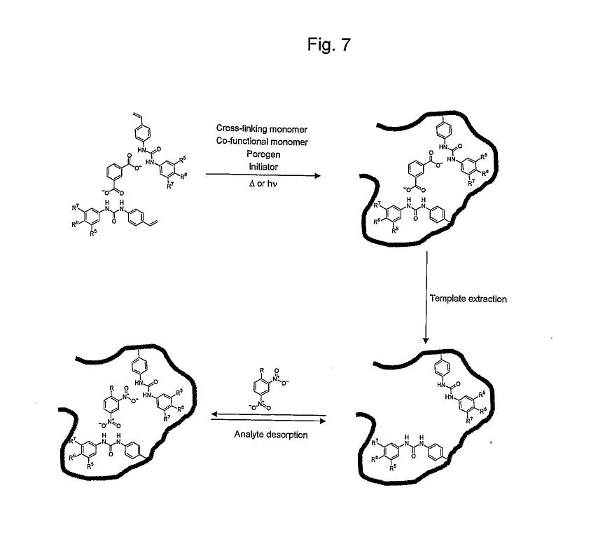

FIG. 7 Schematic illustration of a method according to the invention for the production of molecularly imprinted polymers for selective binding of 1,3-dinitroaromatic compounds;

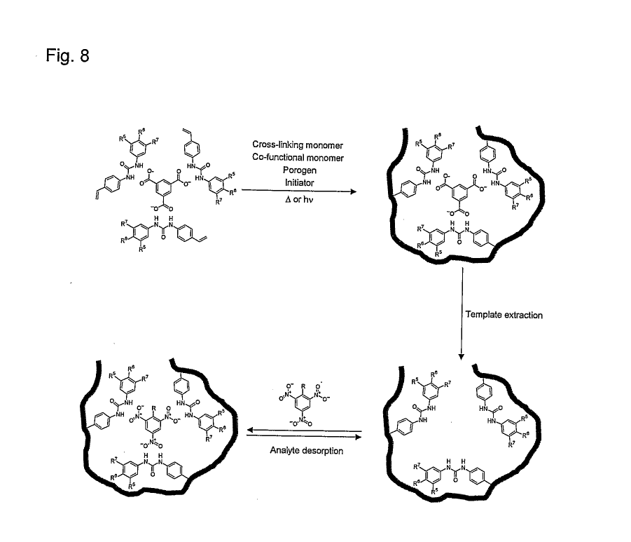

FIG. 8 Schematic illustration of a method according to the invention for the production of molecularly imprinted polymers for selective binding of 1,3,5-trinitroaromatic compounds and similar compounds

### DETAILED DESCRIPTION OF THE INVENTION

The invention relates to a method for preparing molecularly imprinted polymers, which are used for the recognition of target molecules, comprising:

co-polymerising at least one functional monomer and at least one cross-linking monomer in the presence of at least one template in a polymerisation medium to produce a molecularly imprinted polymer bound to the template and, removal of the template from the molecularly imprinted polymer, characterised in that oxyanions are used as template whereby the steric and/or electronic structure of the template is at least partly analogous to the target molecule.

A general description of the technology for producing MIPs may be found in: “Molecularly Imprinted Polymers: Man-Made Mimics of Antibodies and Their Applications in Analytical Chemistry”, Techniques and instrumentation in analytical chemistry, Vol. 23, B. Sellergren (Ed.), Elsevier Science B.V., Amsterdam, 2001

Molecular imprinting typically consists of the following steps: (1) a template compound, which may be the targeted molecule or a structural analogue thereof, is allowed to interact in solution with a selected functional monomer, or monomers, to form a template-monomer complex; (2) the template-monomer complex is co-polymerised with a cross-linking monomer resulting in a polymeric matrix incorporating the template compound; (3) the template compound is extracted from the polymer matrix to form a MIP that can be used for selective binding of the targeted molecule or analogues thereof. Prior to step (3), where the MIP is prepared as a solid polymer (or monolith) it is typically crushed and sieved to obtain a desired size fraction of particulate material.

The polymerisation reaction medium may be homogeneous or heterogeneous. When prepared by either suspension or emulsion polymerisation methods, such crushing and sieving is unnecessary since the particle size can be controlled within the desired limits during the polymerisation process.

Particulate material prepared by any of the aforementioned methods can be packed into a chromatographic or solid phase extraction column and used for chromatographic separation of the template or analogous compound from other components of a mixture, including molecules with similar structures or functionalities.

The binding sites in the molecularly imprinted polymer, exposed by removal of the template compound, will be in a stereo-chemical configuration appropriate for interaction with fresh molecules of the targeted molecule. As a result, the polymer can be used for selective binding of the targeted molecule.

Currently the most widely applied technique to generate molecularly imprinted binding sites is via the ‘non-covalent’ route. This makes use of non-covalent self-assembly of the template compound and functional monomers to form the template-monomer complex, followed by free radical polymerisation in the presence of a cross-linking monomer and finally template compound extraction. Covalent imprinting, in which the template molecule and a suitable monomer or monomers are covalently bound together prior to polymerisation, can also be carried out according to known methods. The binding properties of the MIPs formed by either of the above methods can be examined by re-binding of the template molecule

The polymerisation may be performed in the presence of a pore-forming solvent called a porogen. In order to stabilise the electrostatic interactions between the functional monomers and the template compound the porogen is often chosen from among aprotic solvents of low to moderate polarity. Suitable porogenic solvents are one of chloroform, toluene, acetonitrile or acetonitrile/toluene, tetrahydrofuran and dimethylformamide. Ideally, template compounds exhibit moderate to high solubility in the polymerisation media and these, or their structural analogues, can therefore be used directly using this standard procedure.

The polymerisation medium may contain at least one free radical initiator and the polymerisation may take place either thermally or photochemically.

The target molecule may be nitro-containing compound, such as a nitroaromatic compound, or a lactone.

The nitro-aromatic target compounds for which the MIPs are selective may be chosen from 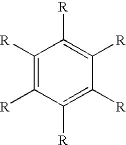
wherein R is H, H₂, NO₂, alkyl with 1-10 carbon atoms or OR¹, wherein R¹ is an alkyl group with 1 to 10 carbon atoms, preferably 1 to 4 carbon atoms, and wherein the ring system may comprise one or more nitro atoms, the formula preferably comprising 1 to 3 of R groups representing NO₂ and 1 to 3 of R groups representing H, H₂ or O R¹.

The formula comprises both six-membered aromatic and non-aromatic rings. When R represents H₂, the system is not aromatic.

Examples of nitroaromatic and related target substances are 1,3 dinitrobenzene (A1), 1,3-DNT (A2), TNT (A3), 1,3,5-trinitrobenzene (A4), picric acid (A5) and picrates and 1,3,5-trinitro-[1,3,5]triazine (Cyclonite, RDX (A6):

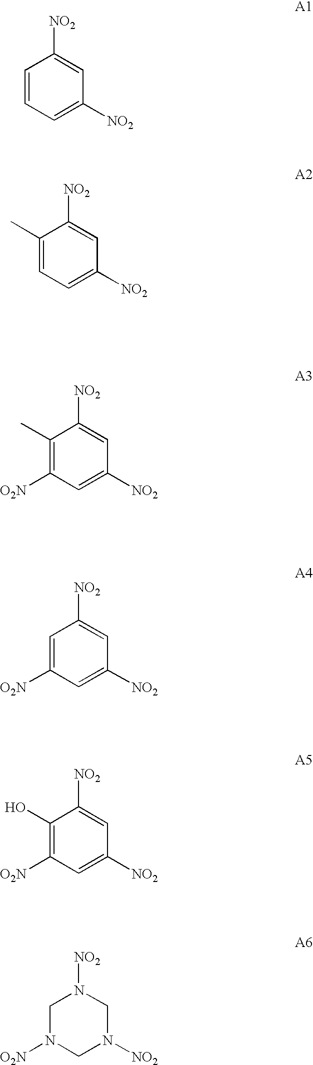

A lactone is a cyclic ester formed by the intramolecular reaction a hydroxyl carboxylic acid molecule with the loss of a water molecule.

The polymer may be prepared using an isosteric and/or isoelectronic template analogue of the nitro-aromatic compound and/or the lactone. In choosing a suitable template, it should be considered that the isosteric structure of the template is almost identical with that of the target molecule. Sometimes it is also preferable that the template is isoelectronic with the target molecule. This is the case in the present invention.

Two molecules are said to be isosteric if they occupy an equal specific volume. Two species are isoelectronic if they have the same electronic configurations.

Such template analogues may be chosen from at least one oxyanion, such as caboxylates, phosphates and/or phosphonates and especially compounds of the formula 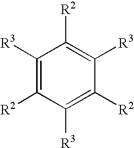
wherein R² is chosen from H and a group COO⁻ M⁺, wherein M⁺ mono-valent cation, such as derived from a primary, secondary or tertiary amine, a quaternary ammonium species or a metal cation, especially an alkali metal cation, and R³ is chosen from H, halogen, alkyl with 1-10 carbon atoms, alkoxy with 1-10 carbon atoms. Especially R³ is an alkyl group with 1-3 carbon atoms, such as methyl. Preferably isophthalates, trimesatesand substituted benzenedicarboxylates and benzenetricarboxylates are used.

The alkali metals may be chosen from lithium, sodium, potassium, rubidium, cesium.

The halogen atom may be chosen from chlorine, fluorine, bromine or iodine.

The choice of oxyanions as templates for the recognition of nitro-containing compounds is especially attractive as these often show the requisite of the partly isosteric structure. The oxyanions are also isosteric to other functionalities, such as lactones. They are furthermore stable under the polymerisation conditions and interact with the functional monomers (for instance urea-based monomers) with the result that binding sites in the polymer matrix can be created. Further, oxyanions are isoelectronic with, amongst other moieties, nitro-groups and lactones.

The advantage of the use of oxyanions of the groups carboxylates, phosphates and/or phosphonates and sulphates is partly the avoidance of explosive nitroaromatic compounds in the production of MIPs and partly the enhanced quality of the MIPs. However, it is theoretically possible to use the above-mentioned nitroaromatic compounds as templates for the production of the MIPs according to the invention.

Particularly preferred is the use of carboxylate anions for the production of molecularly imprinted polymers, whereby urea functional monomers, capable of forming strong bonds to carboxylate anions, are used. The use of carboxylate anions as templates leads to the positioning of urea binding sites in the imprinted polymer matrix. In this way, after the removal of the template, the polymeric binding cavitiespossess complementary functionality to the target molecules.

Especially suitable are isophthalate (T1) and trimesate (T2), with R³ H, or the corresponding toluenes (T3, T4), with R³═CH₃, which are used as templates for the target molecules DNT and TNT, respectively.

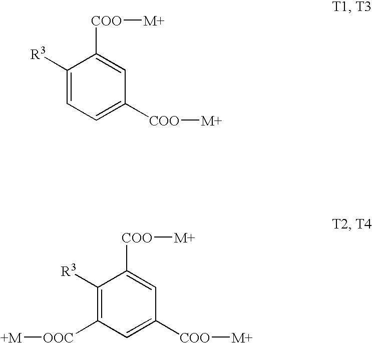

Functional monomers may be chosen from urea compounds with the general structural formula: 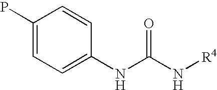
wherein P is a polymerisable group and R⁴ is a single ring or fused ring system comprising 2 or 3 rings, which may comprise oxygen heteroatoms and 1-4 carbonyl groups; phenyl; or R⁴ represents a group with the formula: 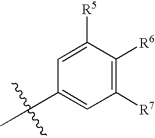
wherein R⁵, R⁶ and R⁷ represent H; alkyl with 1-10 carbon atoms, that may be substituted by one or more halogen atoms; alkoxy with 1-10 carbon atoms, that may comprise one or more halogen atoms; halogen; nitro or amino. The halogen atoms and the alkyl groups may be the same as mentioned above.

Suitable representatives of R⁴ are cumaryl-, anthraquinoyl-, naphthyl-, anthracenyl-, fluorenyl-, 1,2-diphenylazo and indol-7-yl groups, which may be substituted with substituents R⁸ and R⁹, which may be the same as R⁵, R⁶ and R⁷.

Especially preferable as functional monomer are ureas and then specially the use of 1,3-disubstituted ureas.

These exhibit strong interactions with oxyanions such that the polymeric binding sites may be constructed. These compounds may also exhibit chromogenic and/or fluorogenic properties, which can function as signalling elements for the detection of the target molecules.

By chromogenic we understand producing colour. In the current invention, this implies that there is a colour change when the target analyte binds to the functional monomer and/or MIP.

By fluorogenic we understand producing a fluorescence response. In the current invention, this implies that there is a change in the fluorescence behaviour of the monomer and/or MIP on target analyte binding. This can be either a quenching (most common) or enhancement of the fluorescence response.

Ureas are known to bind strongly to oxyanionic species, e.g. carboxylates, phosphates, phosphonates, in a range of solvent environments ranging from low to high polarity, e.g. chloroform to dimethylsulphoxide. The carboxylate anion is isosteric to a number of other functionalities, such as the nitro-group, lactones, etc. These latter functionalities possess only weak Lewis basic properties and, as such, bind only weakly to ureas in solution, although these interactions are useful in the solid phase for directing crystal growth.

Further, the use of nitro-containing compounds as templates is deleterious in free radical polymerisation reactions due to the reactivity of the nitro-function with free radicals. Thus, use of such compounds as templates in molecular imprinting would lead to incomplete polymerisation and, hence, materials of lower quality. Further, the use of aromatic nitro-compounds as explosives adds a significant safety risk to their use as templates in molecular imprinting.

The cross-linking or network monomer may be selected from the group consisting of ethylene glycol dimethacrylate (EDMA), trimethyloylpropane trimethacrylate (TRIM), divinylbenzene (DVB) and pentraerythritol tetraacrylate (PETRA).

MIP, imprinted polymer and polymer matrix are here used as synonyms. Crosslinking monomers and network monomers also mean the same.

Through the use of urea monomers as common functional monomers, of network monomers as for instance ethylenglycoldimethacrylate (EDMA) or divinylbenzene (DVB), and of target molecule analogues as templates, MIPs, exhibiting a high affinity and selectivity towards nitro-containing compounds, especially in the gas phase, can be produced.

FIG. 7 shows a schematic reaction describing one way to perform the method according to the invention for the production of moleculary imprinted polymers with the ability to bind or detect 1,3-dinitroaromatic compounds. Suitable as templates are the dianions of the isophthalic acid (T1). Two equivalents of 1,3-disubstituted monoureas are added as functional monomers. They engage in hydrogen bonding interactions with the carboxylate anions of the template. The free radical polymerisation, thermally or photochemically initiated, occurs in the presence of a cross-linking monomer. These technologies are generally known in the art. The result is a polymer matrix with entrapped templates. Subsequently the templates are released by extraction. This leaves behind a three-dimensional polymer matrix with urea-containing binding sites that exhibit a structure, size and functional group complementarity exactly reflecting the imprint of the template molecule and thus making it possible to recogniserecognise 1,3-dinitroaromatic compounds, for instance 1,3-dinitrobenzene (for R═H) or 2,4-dinitrotoluene (for R═CH3).

FIG. 8 shows the reaction scheme describing another aspect for the production of MIPs for the detection of 1,3,5-trinitroaromatic compounds. Trimesates are here used as templates. Suitable as templates are the trianions of benzene-1,3,5-tricarboxylic acid (T2). Three equivalents of urea monomers are chosen, which engage in hydrogen bonding interactions with the carboxylate anions of the template. Free radical polymerisation, thermally or photochemically initiated, occurs in the presence of a cross-linking monomer. This results in a polymer matrix with entrapped templates. Subsequently, the templates are released by extraction. This leaves behind a three-dimensional polymer matrix with urea-containing binding sites that exhibit a structure, size and functional group complementarity exactly reflecting the imprint of the template molecule and thus making it possible to recognise 1,3,5-trinitroaromatic compounds, for instance 2,4,6-trinitrobenzene (for R═H) or 1,3,5-trinitrotoluene (for R═CH₃). Also the recognition of other explosive compounds such as picrates and 1,3,5-trinitro-[1,3,5]triazine are possible with these imprinted polymers.

Particles comprising MIPs according to the invention, such as the ones described in FIG. 7 and 8 can be packed in columns, capillaries, cartridges, discs or incorporated into membranes. These separation phases are suitable for the very selective and specific binding of nitro-containing target molecules exhibiting a structure and an electron distribution like the template. This is valid both in the gas phase and in apolar solutions.

The invention also relates to a molecularly imprinted polymer (MIP) selective for nitro-containing compounds, such as nitroaromatic compounds, and/or lactones.

The MIPs may be obtained by a method in which a mixture of at least one functional monomer and/or one cross-link monomer and one template is used, whereby oxyanions are used as templates in the polymerizaton, whereby at least one isosteric and/or isoelectronic template analogue of the nitroaromatic compound and/or the lactone are used.

The molecularly imprinted polymer materials—produced according to the method of the invention—can be used for the detection of nitrogenous substances, especially of nitro-aromatic substances, such as, for instance, explosives. Other possible applications are substance specific separations of substances, enrichment, purification, separation or analytical determination of substances in chromatography.

The invention therefore also relates to a method of determining if a sample contains nitro-containing compounds, such as nitro-aromatic compounds, or lactones, comprising:
reacting the sample with a molecularly imprinted polymer selective for nitro-containing compounds, such as nitro-aromatic compounds, or lactones under conditions that would allow binding of nitro-containing compounds, such as nitro-aromatic compounds, or lactones present in the sample to the molecularly imprinted polymer; and
evaluating whether the molecularly imprinted polymer has bound any nitro-containing compounds, such as nitro-aromatic compounds, or lactones, wherein an evaluation resulting in observation of binding to the molecularly imprinted polymer by nitro-containing compounds, such as nitro-aromatic compounds, or lactones indicates that the sample contains nitro-containing compounds, such as nitro-aromatic compounds, or lactones; and optionally measuring the amount of nitro-containing compounds, such as nitro-aromatic compounds, or lactones bound to the molecularly imprinted polymer.

MIPs, produced according to the method of the invention, have a high affinity and selectivity for the recognition of nitro-containing compounds, such as nitro-aromatic compounds, and lactones.

The invention also relates to a kit, comprising a molecularly imprinted polymer selective for nitro-containing compounds, such as nitroaromatic compounds, and/or lactones; and instructions for using the molecularly imprinted polymer to perform at least one of detecting, quantifying, and separating nitro-containing compounds, such as nitroaromatic compounds, and/or lactones in a sample.

In addition the invention relates to the use of isosteric and/or isoelectronic oxyanions, especially benzenedicarboxylates and benzenetricarboxylates, isophthalates and trimesates for the production of MIPs for the recognition of nitro-containing compounds, especially nitroaromatic compounds, and lactones.

All the details regarding methods for preparing the MIPs according to the invention are equally applicable to all other aspects of the invention, e.g. MIPs as such; a method of determining if a sample contains nitro-containing compounds, such as nitro-aromatic compounds, or lactones; a kit comprising the MIPs of the invention or the use of isosteric and/or isoelectronic oxyanions for the recognition of nitro-containing compounds, especially nitroaromatic compounds, and lactones. Thus, the target molecule, the template molecule, the functional monomers and the cross-linking monomers, etc., may be chosen from the ones mentioned above.

While the invention has been described in relation to certain disclosed embodiments, the skilled person may foresee other embodiments, variations, or combinations that are not specifically mentioned but are nonetheless within the scope of the appended claims. The invention will now be described by way of the following non-limiting examples. All the publications mentioned herein are enclosed by reference.

### EXAMPLE 1

### Synthesis of Strong-Binding Urea Functional Monomers

These monomers are prepared in good to excellent yield in one step, either from a polymerisable aniline and a non-polymerisable phenyl isocyanate or vice versa (see FIG. 1) (Hall et al., J. Org. Chem. 2005, 70, 1732-1736). The polymerisable isocyanate shown may be prepared in good yield from 4-toluic acid in four synthetic steps. (Lübke et al., J. Am. Chem. Soc. 1998, 120, 13342-13348)

In the simplest case, R⁵═R⁶═R⁷═H, i.e. 1-(4-vinylphenyl)-3-phenyl urea. The Lewis acidity of the urea functional monomer may be tuned by variation of the nature of the substituents R⁵, R⁶ and R⁷. Thus, when these substituents are electron-donating groups, e.g. methyl, methoxy, amino, etc., the Lewis acidity and, hence, the hydrogen bonding ability of the functional monomers, is reduced in comparison to the parent compound U1. Conversely, when substituents R⁵, R⁶ and R⁷ are electron-withdrawing groups, e.g. halide, nitro-, trifluoromethyl, etc., the Lewis acidity and hydrogen bonding ability of the monomers are enhanced in comparison to U1.

Some non-limiting examples for strong binding polymerisable ureas, U1, U2 and U3, are shown in FIG. 2.

### EXAMPLE 2

### Synthesis of Chromogenic Urea Functional Monomers

These monomers are prepared in good to excellent yield in one step, either from a polymerisable aniline and a non-polymerisable phenyl isocyanate or vice versa. On binding with the template molecule or the target analyte, a detectable change in the colorimetric properties of the monomer units are observed, both when the monomers are in solution and when incorporated into an imprinted polymer matrix. This leads the way towards imprinted polymer colorimetric sensors. Some non-limiting examples, U3, U4, U5 and U6 are shown in FIG. 3.

### EXAMPLE 3

### Synthesis of Fluorogenic Urea Functional Monomers

These monomers are prepared in good to excellent yield in one step, either from a polymerisable aniline and a non-polymerisable phenyl isocyanate or vice versa. On binding with the template molecule or the target analyte, a detectable change in the fluorimetric properties of the monomer units are observed, both when the monomers are in solution and when incorporated into an imprinted polymer matrix. This leads the way towards imprinted polymer fluorimetric sensors. Some non-limiting examples, U7, U8 and U9, are shown in FIG. 8.

In addition to the above fluorogenic mono-ureas, we also include the novel indole-containing mono-ureas. These monomers are formed in two steps, from an appropriate 7-nitroindole, in moderate to good overall yield. The acidic indole NH proton is properly positioned such that a third hydrogen bond to template and analyte molecules is possible. Thus, in addition to the fluorescence activity of the indole moiety, an enhancement in binding strength is observed with for instance oxyanions or nitro-containing compounds. Some non-limiting examples, U11 and U12, are shown in FIG. 5.

### EXAMPLE 4

### Synthesis of a Cross-Linking Urea Functional Monomer

This monomer, U13 (see FIG. 6) is prepared from 4-vinylaniline and 4-vinylphenyl isocyanate in good yield in one synthetic step. The monomer can be used to introduce extra rigidity into the polymeric binding site. Further, the use of this monomer as both binding element and cross-linker allows the introduction of other functional monomers in place of a portion of the “normal” cross-linking monomer, e.g. monomers which enhance the recognition process or introduce hydrophilicity to the polymer matrix, without causing a deleterious effect on either the binding site rigidity or the thermal and mechanical stability of the imprinted polymers.

### EXAMPLE 5

### Synthesis of Materials for the Selective Recognition of Dinitro-Aromatic Compounds

The target analytes, 1,3-dinitrobenzene (A1, R═H) or 2,4-dinitrotoluene (A2, R═CH₃), are substituted as templates in the imprinting protocol by the dianion of isophthalic acid (T1). Two equivalents of a 1,3-mono-urea functional monomer, chosen from those detailed in Examples 1-4, are used to coordinate, through hydrogen bonds, to the two carboxyanion groups of the template (see FIG. 7).

Free-radical polymerisation (thermal or photochemical initiation) in the presence of an excess of cross-linking monomer, followed by solvent extraction of the anionic template in a Soxhlet apparatus, leads to a three-dimensional polymer matrix with urea-containing binding pockets, whose shape, size and functional group directionality are perfectly tailored for the reuptake of the template molecule AND for the recognition of the analytes, A1 and A2.

In a typical procedure, isophthalic acid (1 mmol, 0.167 g), triethylamine (2 mmol, 0.20 g), functional monomer U1 (2 mmol, 0.75 g) and the cross-linking monomer ethylene glycol dimethacrylate (EDMA) (20 mmol, 3.96 g) are dissolved in dimethylformamide (5.6 mL). The azo-initiator, 2,2′-azobis-(2,4-dimethylvaleronitrile) (ABDV) (1% w/w total monomers,) is then added to the solution. After dissolution of ABDV, the solution is transferred to a polymerisation tube. The tube and its contents are cooled on ice and then purged with dinitrogen for ten minutes to remove dissolved oxygen from the solution. Thereafter, the polymerisation tube is capped and then placed in a pre-heated water bath, thermostatted at 45° C., thus initiating the polymerisation. Polymerisation is allowed to continue for 48 hours at 45° C., whereupon the polymerisation tube is broken and the monolithic imprinted polymer removed. The monolithic polymer is then roughly broken into smaller pieces and the template molecule (the salt formed between isophthalic acid and triethylamine) is removed from the imprinted polymer by continuous extraction with methanol (250 mL) in a Soxhlet apparatus for 24 hours. The extracted polymer may then be crushed and sized to give the desired particles, depending on the application for which they are intended.

The polymer was capable of selectively and strongly binding the targeted nitro-aromatic compounds from apolar media, such as from the gas phase or apolar solvents.

### EXAMPLE 6

### Synthesis of Materials for the Selective Recognition of Trinitro-Aromatic Compounds

In this example, the target analytes, 1,3,5-trinitrotoluene (A3, R═CH₃) or 2,4,6-trinitrobenzene (A4, R═H), are substituted as the template in the imprinting protocol by the trianion of benzene-1,3,5-tricarboxlic acid (T2). Three equivalents of urea functional monomer are utilised to coordinate, through hydrogen bonds, to the three carboxyanion groups of the template (see FIG. 7).

Free-radical polymerisation (thermal or photochemical initiation) in the presence of an excess of cross-linking monomer, followed by extraction of the anionic template, leads to a three-dimensional polymer matrix with urea-containing binding pockets, whose shape, size and functional group directionality are perfectly tailored for the reuptake of the template molecule AND for the recognition of the analytes, namely A3 and A4. Further, recognition of other explosive compounds, such as picrates (A5) and 1,3,5-trinitro-[1,3,5]triazinane (A6), is possible within these imprinted polymers.

In a typical procedure, trimesic acid (1 mmol, 0.21 g), triethylamine (3 mmol, 0.30 g), functional monomer U1 (3 mmol, 1.12 g) and the cross-linking monomer ethylene glycol dimethacrylate (EDMA) (20 mmol, 3.96 g) are dissolved in dimethylformamide (5.6 mL). The azo-initiator, 2,2′-azobis-(2,4-dimethylvaleronitrile) (ABDV) (1% w/w total monomers, 51 mg) is then added to the solution. After dissolution of ABDV, the solution is transferred to a polymerisation tube. The tube and its contents are cooled on ice and then purged with dinitrogen for ten minutes to remove dissolved oxygen from the solution. Thereafter, the polymerisation tube is capped and then placed in a pre-heated water bath, thermostatted at 45° C., thus initiating the polymerisation. Polymerisation is allowed to continue for 48 hours at 45° C., whereupon the polymerisation tube is broken and the monolithic imprinted polymer removed. The monolithic polymer is then roughly broken into smaller pieces and the template molecule (the salt formed between isophthalic acid and triethylamine) is removed from the imprinted polymer by continuous extraction with methanol (250 mL) in a Soxhlet apparatus for 24 hours. The extracted polymer may then be crushed and sized to give the desired particles, depending on the application for which they are intended.

The polymer was capable of selectively and strongly binding the targeted nitro-aromatic compounds from apolar media, such as from the gas phase or apolar solvents.

1.-17. (canceled)

18. A molecularly imprinted polymer used for the recognition of target molecules selected from nitro-containing compounds and/or lactones, wherein said molecularly imprinted polymer is obtainable by:
co-polymerising at least one functional monomer and at least one cross-linking monomer in the presence of at least one template in a polymerisation medium to produce a molecularly imprinted polymer bound to the template and removing the template from the molecularly imprinted polymer,
characterised in that oxyanions are used as template whereby the steric and electronic structure of the template is at least partly analogous to the target molecule.

19. A molecularly imprinted polymer according to claim 18, wherein the nitro-containing compound is a nitro-aromatic compound.

20. A molecularly imprinted polymer according to claim 19, wherein the nitro-aromatic compounds are chosen from 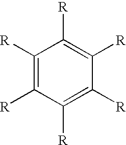
wherein R is H, H₂,NO₂, alkyl with 1-10 carbon atoms or OR¹, wherein R¹ is an alkyl group with 1 to 10 carbon atoms, preferably 1 to 4 carbon atoms, and wherein the ring system may comprise one or more nitro atoms, the formula preferably comprising 1 to 3 of R groups representing NO₂ and 1 to 3 of R groups representing H, H₂ or O R¹.

21. A molecularly imprinted polymer according to claim 20, wherein the nitro-aromatic compound is chosen from 1,3-dinitrobenzene, 1,3-dinitrotoluene, trinitrotoluene, 1,3,5-trinitrobenzene, picrates or 1,3,5-trinitro-[1,3,5]triazine (Cyclonite, RDX).

22. A molecularly imprinted polymer according to claim 21, characterised by the template being an isosteric and/or isoelectronic template analogue of the nitro-containing compound and/or the lactone.

23. A molecularly imprinted polymer according to claim 18, wherein the template an isosteric and/or isoelectronic template analogue of the nitro-containing compound and/or the lactone.

24. A molecularly imprinted polymer according to claim 19, wherein the template an isosteric and/or isoelectronic template analogue of the nitro-containing compound and/or the lactone.

25. A molecularly imprinted polymer according to claim 20, wherein the template an isosteric and/or isoelectronic template analogue of the nitro-containing compound and/or the lactone.

26. A molecularly imprinted polymer according to claim 25, characterised in that the template is chosen from oxyanions, such as carboxylates, phosphates and/or phosphonates and especially isophthalates, trimesates, benzenedicarboxylates and benzenetricarboxylates, as an isosteric analogue of nitro-containing compound as a targets.

27. A molecularly imprinted polymer according to claim 24, characterised in that the template is chosen from oxyanions, such as carboxylates, phosphates and/or phosphonates and especially isophthalates, trimesates, benzenedicarboxylates and benzenetricarboxylates, as an isosteric analogue of nitro-containing compound as a targets.

28. A molecularly imprinted polymer according to claim 23, characterised in that the template is chosen from oxyanions, such as carboxylates, phosphates and/or phosphonates and especially isophthalates, trimesates, benzenedicarboxylates and benzenetricarboxylates, as an isosteric analogue of nitro-containing compound as a targets.

29. A molecularly imprinted polymer according to claim 18, characterised in that the template is chosen from oxyanions, such as carboxylates, phosphates and/or phosphonates and especially isophthalates, trimesates, benzenedicarboxylates and benzenetricarboxylates, as an isosteric analogue of nitro-containing compound as a targets.

30. A molecularly imprinted polymer according to claim 18, wherein the functional monomer is selected from monomer chosen from compounds of 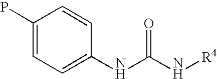
wherein P is a polymerisable group and R⁴ is a single ring or fused ring system comprising 2 or 3 rings, which may comprise oxygen heteroatoms and 1-4 carbonyl groups; phenyl; or R⁴ represents a group with the formula: 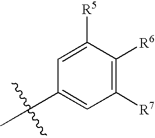
wherein R⁵, R⁶ and R⁷ independently represent H; alkyl with 1-10 carbon atoms, that may be substituted by one or more halogen atoms; alkoxy with 1-10 carbon atoms, that may comprise one or more halogen atoms; halogen; nitro or amino. The halogen atoms and the alkyl groups may be the same as mentioned above.

31. A molecularly imprinted polymer according to claim 30, wherein the functional monomer is selected from 1,3-disubstituted ureas, such as 1-(4-vinylphenyl)-3-phenylurea, as a functional monomer.

32. A molecularly imprinted polymer according to claim 18, wherein the cross-link monomer is selected from the group consisting of ethylene glycol dimethacrylate (EDMA), trimethyloylpropane trimethacrylate (TRIM), divinylbenzene (DVB) and pentraerythritol tetraacrylate (PETRA).

33. A molecularly imprinted polymer according to claim 18, wherein the reaction medium is homogeneous or tetrogeneous.

34. A method of determining if a sample contains nitro-containing compounds, such as nitro-aromatic compounds, or lactones, comprising:
reacting the sample with a molecularly imprinted polymer according to any one of claims 1-10 selective for nitro-containing compounds, such as nitro-aromatic compounds, or lactones under conditions that would allow binding of nitro-containing compounds, such as nitroaromatic compounds, or lactones present in the sample to the molecularly imprinted polymer; and
evaluating whether there has been binding to the molecularly imprinted polymer by any nitro-containing compounds, such as nitro-aromatic compounds or lactones, wherein an evaluation resulting in observation of binding to the molecularly imprinted polymer by nitro-containing compounds, such as nitro-aromatic compounds, or lactones indicates that the sample contains nitro-containing compounds, such as nitroaromatic compounds, or lactones; and optionally measuring the amount of nitro-containing compounds, such as nitro-aromatic compounds or lactones bound to the molecularly-imprinted polymer.

35. A kit, comprising a molecularly imprinted polymer prepared according to claim 18, selective for nitro-containing compounds, especially nitroaromatic compounds, and/or lactones; and instructions for using the molecularly imprinted polymer to perform at least one of detection, quantification, and separation of nitro-containing compounds, especially nitroaromatic compounds, or lactones in a sample.

36. Use of a molecularly imprinted polymer according to claim 18, in a chemical sensor.

37. Use of a molecularly imprinted polymer according to claim 18, in extraction of a target molecule.

38. Use of a molecularly imprinted polymer according to claim 18, in separation of a target molecule selected from nitro-containing compounds and lactones.

39. Use of isosteric and/or isoelectronic oxyanions for the production of MIPs for recognition of nitro-containing compounds and lactones.

40. The use according to claim 39, wherein the oxyanion is selected from isoelectronic and/or isosteric benzene di- or tricarboxylates, isophthalates and trimesates for the production of MIPs for detection of nitro-containing compounds, especially of nitro-aromatic compounds.

41. The use according to claim 39, wherein the nitro-containing compounds include nitroaromatic compounds.
